(19) United States
(12) Patent Application Publication
(10) Pub. No.: US 2007/0292499 A1
(43) Pub. Date: Dec. 20, 2007

(54) Processes for the Preparation of Solid Dosage Forms of Amorphous Valganciclovir Hydrochloride

(76) Inventors: Romi Barat Singh, Varanasi (IN); Vishnubhotla Nagaprasad, Hyderabad (IN); Nidhi Singh, New Delhi, IN (US)

Correspondence Address: RANBAXY INC., 600 COLLEGE ROAD EAST, SUITE 2100, PRINCETON, NJ, 08540, US

(21) Appl. No.: 10/598,546

(22) PCT Filed: Mar. 10, 2005
(86) PCT No.: PCT/IB05/00615

§ 371 Date: Jun. 4, 2007

(30) Foreign Application Priority Data
Mar. 10, 2004 (IN) 410/DEL/2004

(51) Int. Cl. A61K 9/26; A61K 31/522; A61K 9/48; A61P 31/22

(52) U.S. Cl. 424/452; 424/465; 514/258.1

(57) ABSTRACT

The present invention relates to a process for the preparation of solid dosage forms of amorphous valganciclovir hydrochloride by dry method.

### FIELD OF THE INVENTION

The present invention relates to a process for the preparation of solid dosage forms of amorphous valganciclovir hydrochloride by dry method.

### BACKGROUND OF THE INVENTION

Valganciclovir hydrochloride, a prodrug of gancilcovir, is used in the treatment of cytomegalovirus (CMV) retinitis in patients with acquired immunodeficiency syndrome (AIDS) and for the prevention of CMV disease in kidney, heart and for kidney-pancrease transplant patients who are at high risk. Valganciclovir hydrochloride is hydrochloride salt of the L-valyl ester of ganciclovir. Chemically, it is L-Valine, 2-[(2-amino-1,6-dihydro-6-oxo-9H-purin-9-yl)methoxy]-3-hydroxypropyl ester, monohydrochloride. It is available as tablets under the brand name Valcyte® containing crystalline valganciclovir hydrochloride.

U.S. Pat. No. 6,083,953 discloses valganciclovir hydrochloride and processes for its preparation. Therein it is set forth that valganciclovir hydrochloride was developed with a view to improve the bioavailability of gancilcovir, which has a poor bioavailability when administered orally. The higher oral doses of ganciclovir are required to achieve a therapeutic concentration in the blood. Valganciclovir hydrochloride when administered orally is reported to have better bioavailability than ganciclovir given orally.

It is known that the amorphous form of a drug may have certain advantages over the crystalline form, for example, amorphous form can be more soluble or can have a higher rate of solubility in water than the crystalline form and consequently the drug may show improved bioavailability, for example, due to the faster dissolution of the drug in the gastrointestinal fluid. It is with this view that we have prepared dosage forms comprising valganciclovir hydrochloride in which valganciclovir hydrochloride is present in the amorphous form. Without being bound by any theory, these dosage forms may further improve the oral bioavailability of valganciclovir and ultimately that of ganciclovir.

However, it has been found that amorphous valganciclovir hydrochloride as such is very fine and fluffy material, with relatively low bulk and tap density. This property can make it difficult to formulate into a dosage form with uniformity of weight, hardness, and other desirable tablet properties. Wet granulation needs to be avoided, as addition of a solvent along with subsequent removal in way of drying the granules at elevated temperatures may convert the amorphous form to crystalline form.

The direct compression technique may not be desirable for a drug with a bulk density less than 0.2 g/cc, due to poor flow of the material leading to non-uniform die-fill and subsequent weight variation. However, direct compression could be a method of choice when the amorphous valganciclovir hydrochloride is so processed that it has a bulk density of at least 0.2 g/cc or more. This can be achieved during chemical manufacturing by variation in the processing parameters or by a physical process of compaction and milling.

There is a need for simple method of production, which does not require wet granulation with organic solvents or water and do not require an additional step of drying.

### SUMMARY OF THE INVENTION

We hereby report processes for the preparation of solid dosage forms comprising amorphous valganciclovir hydrochloride by a dry process which may be, for example, dry granulation or direct compression. The processes give dosage forms with uniformity of weight, sufficient hardness and friability.

In one general aspect, the present disclosure relates to dry processes for the preparation of solid dosage forms comprising amorphous valganciclovir hydrochloride and one or more pharmaceutically acceptable excipient(s).

In another general aspect, herein are provided processes for the preparation of solid dosage forms of amorphous valganciclovir hydrochloride wherein the processes comprise mixing amorphous valganciclovir hydrochloride with one or more pharmaceutically acceptable excipient(s) and forming into solid dosage forms.

In another general aspect, herein are provided processes for the preparation of solid dosage forms of amorphous valganciclovir hydrochloride wherein the processes comprise mixing amorphous valganciclovir hydrochloride with one or more pharmaceutically acceptable excipient(s) and compressing into a tablet.

In another general aspect, herein are provided processes for the preparation of solid dosage forms of amorphous valganciclovir hydrochloride wherein the processes comprise compacting valganciclovir hydrochloride alone or mixed with one or more pharmaceutically acceptable excipient(s) by, for example, roller compaction or slugging; sizing the compacts into granules by milling; optionally mixing the granules with one or more of pharmaceutically acceptable excipients and forming a solid dosage form.

In another general aspect, herein are provided processes for the preparation of solid dosage forms of amorphous valganciclovir hydrochloride wherein the processes comprise compacting valganciclovir hydrochloride alone or mixed with one or more pharmaceutically acceptable excipient(s) by, for example, roller compaction or slugging; sizing the compacts into granules by milling; optionally mixing the granules with one or more of pharmaceutically acceptable excipients and compressing into a tablet.

In another general aspect, herein are provided methods of administering amorphous valganciclovir hydrochloride to a patient in need thereof as solid dosage forms prepared by a dry process.

### BRIEF DESCRIPTION OF THE DRAWINGS

FIG. 1 is a set of x-ray diffractogram of tablets of Example 1 recorded upon production and after storage for two months at 40° C. and 75% relative humidity.

### DETAILED DESCRIPTION OF THE INVENTION

The present invention provides solid dosage forms comprising valganciclovir hydrochloride in amorphous form prepared by a dry process. The process is simple and economical as it does not require any solvents, as in the case of wet granulation processes, the most commonly followed process, which requires additional steps of drying the granules. The amorphous form resists conversion to a crystalline form by following the dry processes as described herein.

The term “solid dosage form” as used herein includes granules, tablets or coated tablets and capsules filled with granules or tablets prepared as per the embodiments described herein. Particularly suitable solid dosage forms are tablets.

The amorphous valganciclovir hydrochloride in the solid dosage form is present in a therapeutically effective amount. Typically, the drug may comprise from about 1 mg to about 1000 mg, for example, from about 50 mg to about 800 mg. The amorphous valganiclovir can be prepared by methods described in Indian Patent Application No. 1052/DEL/2003 filed 28 Aug. 2003.

Additionally, other drugs in a therapeutically effective amount can also be combined with the present dosage forms.

The pharmaceutically acceptable excipients are those known to the skilled in the art and may be selected from fillers, binders, disintegrants, glidant and lubricants. These excipients may be present intragranularly or extragranularly or both.

The fillers may be selected from microcrystalline cellulose, mannitol, sucrose, lactose, dextrose, calcium carbonate, sorbitol and the like. The filler may be present in an amount of about 15% to about 60%, for example, from about 20% to 40% by weight of the dosage form.

The binders may be selected from polyvinylpyrrolidone, hydroxypropylcellulose, hydroxypropylmethylcellulose, starch and starch based binders, gelatin, gums and the like. The binder may be present in an amount of about 0.1% to about 10%, for example, from about 1% to about 5% by weight of the dosage form.

The disintegrant may be selected from crospovidone, croscarmellose sodium, starch, hydroxypropylcellulose, hydroxypropylmethylcellulose, gums, sodium starch glycolate and the like. The disintegrant may be present in an amount of about 1% to about 40%, for example, from about 2% to about 20% by weight of the dosage form.

The lubricants and glidants may be selected from talc, colloidal silicon dioxide, magnesium stearate, stearic acid and sodium stearyl fumarate. These may be present in an amount of about 0.1 % to about 2% by weight of the dosage form.

The compaction of the drug or the mixture comprising the drug and excipient(s) into compacts may be carried out by slugging or by roller compaction, particularly suitable is roller compaction.

The roller compactor functions by uniformly applying pressure on a mixed powder blend by passing the blend between two counter-rotating rollers. The pressure imparted on the blend by the rollers compresses the powder into a compact, such as a sheet or ribbon, which is typically milled to produce granules.

In one general aspect of the process, the valganciclovir hydrochloride can be mixed with one or more of pharmaceutical excipients such as filler, binder, disintegrant and lubricant described above in a suitable blender. The mixing time can vary from about 10 to 60 minutes. The resultant blend can either be directly compressed into solid dosage form or compacted by roller compaction.

The resultant blend for compaction is subsequently transferred to a roller compactor in a known manner. The roller speed, roller gap width and force of compaction are then adjusted and the blend is fed through the roller compactor. The typical force and other conditions can be easily selected and adjusted by those skilled in the art. For example, the compaction pressure may be between 25 to 75 bar, for example, between 35 to 55 bar. For maintaining the steady output of the compact material from the roller compactor, the rollers may be rotated at a speed of between 1 to 20 rpm, for example, between 2 to 10 rpm, or between 3 to 5 rpm. When in contact with the counter rotating rollers of the roller compactor, the compression force imparted on the blend by rollers converts the powdered form into a ribbon or compaction sheet. This compact sheet is fed to a mill, such as an oscillating mill, fitted with a screen. The screen can be selected with variable hole diameters depending upon the size of the granules required. After passing through the mill and the screen, the compact is converted into granules of the desired particle size distribution.

The granules obtained as above may be filled into capsules or packed in a sachet. The granules can also be mixed with one or more of pharmaceutically acceptable excipients and compressed into tablets.

As an alternative to roller compaction, slugging may be used for preparing solid dosage forms such as a tablet. The process is simple, low cost and effective. Slugging may be carried out by means of a tablet press. The drug either alone or mixed with other excipients is precompressed on a heavy duty press. The slug so formed is milled into granules and recompressed into tablet. The granules may also optionally be mixed with other extragranular excipients prior to compression into a tablet.

Both the processes, i.e., roller compaction and slugging generate fines during the milling step. A portion of these fines can be mixed with granules and compressed into a tablet or can be easily recycled by collecting them and again compacted.

The tablet must be of sufficient hardness to withstand the packaging, transport or coating process without chipping or breaking. The hardness of the tablets can be measured by known methods. The hardness should not be so high that it adversely affects disintegration and dissolution rates of the tablets. Preferably, the hardness of these amorphous valganciclovir hydrochloride tablets is about 10 kP to about 30 kP, for example, about 15 kP to about 25 kP.

Another measure of durability of the tablet is the test for friability. A friability values of about 1% is acceptable, but friability below about 0.8% is particularly suitable. The tablets prepared as per the process can have friability of less than 0.8%, for example, less than 0.5%.

In one embodiment, processes provided herein comprise mixing amorphous valganciclovir hydrochloride with one or of pharmaceutically acceptable excipients and compressing the blend into a tablet.

In one embodiment, processes provided herein comprise compacting amorphous valganciclovir hydrochloride alone or mixed with a lubricant using roller compactor; milling and sizing the compacts into granules with a desired particle size distribution; mixing with extragranular pharmaceutically acceptable excipient(s) and compressing into a tablet using appropriate tooling.

In another embodiment, processes herein comprise mixing amorphous valganciclovir hydrochloride, filler, binder, disintegrant and lubricant and compacting the mixture using roller compactor; milling and sizing the compacts into granules with a desired particle size distribution; mixing with lubricant and compressed into a tablet using appropriate tooling.

In still another embodiment, processes provided herein comprise mixing amorphous valganciclovir hydrochloride, filler, binder, disintegrant and lubricant and compacting the mixture using roller compactor; milling and sizing the compacts into granules with a desired particle size distribution; mixing with one or more of filler, binder, disintegrant and lubricant and compressed into a tablet using appropriate tooling.

In yet another embodiment, processes provided herein comprise compacting amorphous valganciclovir hydrochloride alone or mixed with one or more of filler, binder, disintegrant and lubricant, milling and sizing the compacts into granules with a desired particle size distribution and filling into a capsule dosage form.

In still another embodiment, processes provided herein comprise compacting amorphous valganciclovir hydrochloride alone or mixed with one or of pharmaceutically acceptable excipients by slugging; milling and sizing the slugs into granules with a desired particle size distribution; optionally mixing the granules with one or more of filler, binder, disintegrant and lubricant and compressed into a tablet.

When the solid dosage form is a tablet then it may additionally be coated with coating compositions like Opadry® AMB (with or without a non aqueous subcoat) sold by Colorcon to impart moisture protection on stability. Such a coating may comprise about 3-10% w/w of the tablet. The tablet may also be coated with coating compositions like Opadry® or Lustreclear® sold by Colorcon using non-aqueous or aqueous systems, preferably non-aqueous system to impart aesthetic appeal as well as a barrier to the external environment. Such a coating may comprise about 3-10% w/w of the tablet.

The invention described herein can further be illustrated by the following examples but these do not limit the scope of the invention.

### EXAMPLE 1

| | Quantity |
|---|---|
| Ingredients | (mg) |
| Valganciclovir hydrochloride (amorphous) | 496.3 |
| eq. to 450 mg of valganciclovir | |
| Microcrystalline cellulose | 159.95 |
| Cross-linked polyvinylpyrrolidone | 21.0 |
| Polyvinylpyrrolidone | 14.0 |
| Magnesium stearate | 8.75 |
| Total | 700 |

Procedure:
Valganciclovir hydrochloride (amorphous) was mixed in a blender with microcrystalline cellulose, cross-linked polyvinylpyrrolidone, polyvinylpyrrolidone and magnesium stearate and compressed into tablet using appropriate tooling.

### EXAMPLE 2

| | Quantity |
|---|---|
| Ingredients | (mg) |
| Intragranular | |
| Valganciclovir hydrochloride (amorphous) | 496.3 |
| eq. to 450 mg of Valganciclovir | |
| Microcrystalline cellulose | 159.95 |
| Cross-linked polyvinylpyrrolidone | 21.0 |
| Polyvinylpyrrolidone | 14.0 |
| Magnesium stearate | 3.50 |
| Extragranular | |
| Magnesium stearate | 5.25 |
| Total | 700 |

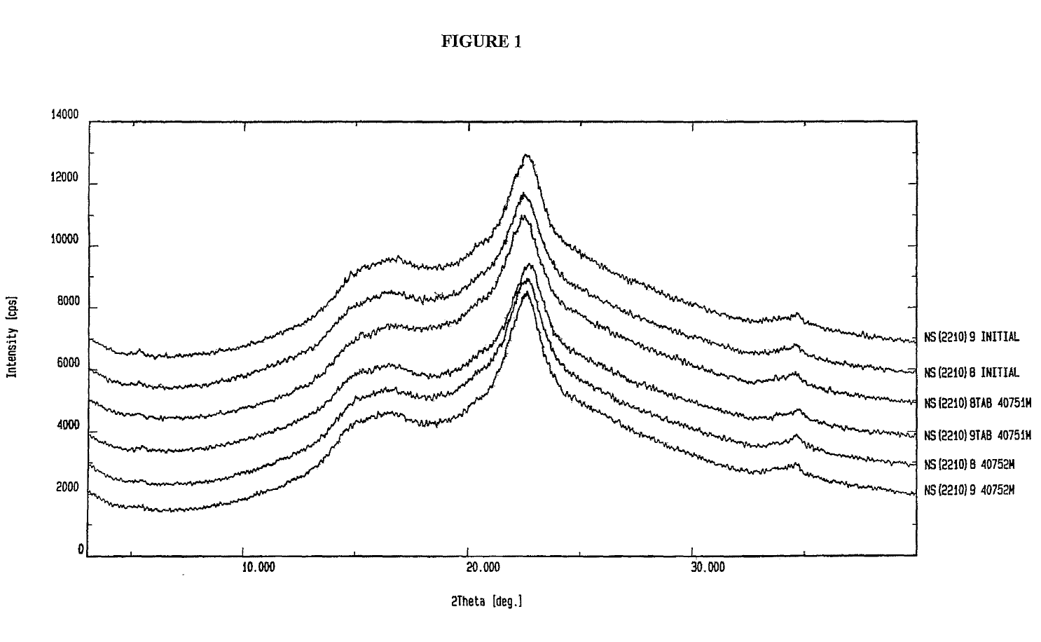

Procedure:
Valganciclovir hydrochloride (amorphous) was mixed with microcrystalline cellulose, cross-linked polyvinylpyrrolidone, polyvinylpyrrolidone and magnesium stearate and compacted with a roller compactor. The compacts were sized into granules by milling, mixed with magnesium stearate and compressed into tablet using appropriate tooling.
The tablets of Example 1 were subjected to accelerated stability testing. The tablets were kept at 40° C. and 75% relative humidity for two months. The XRD data at the end of the two months period showed no change in amorphous nature in comparison to the initial scan of the amorphous valganciclovir hydrochloride, as shown in FIG. 1.

### EXAMPLE 3

| | Quantity |
|---|---|
| Ingredients | (mg) |
| Intragranular | |
| Valganciclovir hydrochloride (amorphous) | 496.3 |
| eq. to 450 mg of Valganciclovir | |
| Magnesium stearate | 3.50 |
| Extragranular | |
| Microcrystalline cellulose | 159.95 |
| Cross-linked polyvinylpyrrolidone | 21.0 |
| Polyvinylpyrrolidone | 14.0 |
| Magnesium stearate | 5.25 |
| Total | 700 |

Procedure:
Valganciclovir hydrochloride (amorphous) and magnesium stearate were mixed in a blender and compacted using a roller compactor. The compacts were sized into granules by milling, mixed with microcrystalline cellulose, cross-linked polyvinylpyrrolidone, polyvinylpyrrolidone and magnesium stearate and compressed into tablet using appropriate tooling.

### EXAMPLE 4

| | Quantity |
|---|---|
| Ingredients | (mg) |
| Intragranular | |
| Valganciclovir hydrochloride (amorphous) | 496.3 |
| eq. to 450 mg of Valganciclovir | |
| Microcrystalline cellulose | 79.975 |
| Cross-linked polyvinylpyrrolidone | 21.0 |
| Polyvinylpyrrolidone | 14.0 |
| Magnesium stearate | 3.50 |
| Extragranular | |
| Microcrystalline cellulose | 79.975 |
| Magnesium stearate | 5.25 |
| Total | 700 |

Procedure:
Valganciclovir hydrochloride (amorphous) was mixed with microcrystalline cellulose, cross-linked polyvinylpyrrolidone, polyvinylpyrrolidone and magnesium stearate and compacted with a roller compactor. The compacts were sized into granules by milling, mixed with microcrystalline cellulose and magnesium stearate and compressed into tablet using appropriate tooling.

### EXAMPLE 5

| | Quantity |
|---|---|
| Ingredients | (mg) |
| Intragranular | |
| Valganciclovir hydrochloride (amorphous) | 496.3 |
| eq. to 450 mg of Valganciclovir | |
| Microcrystalline cellulose | 79.975 |
| Cross-linked polyvinylpyrrolidone | 10.5 |
| Polyvinylpyrrolidone | 14.0 |
| Magnesium stearate | 3.50 |
| Extragranular | |
| Microcrystalline cellulose | 79.975 |
| Cross-linked polyvinylpyrrolidone | 10.5 |
| Magnesium stearate | 5.25 |
| Total | 700 |

Procedure:
Valganciclovir hydrochloride (amorphous) was mixed with microcrystalline cellulose, cross-linked polyvinylpyrrolidone, polyvinylpyrrolidone and magnesium stearate and compacted with a roller compactor. The compacts were sized into granules by milling, mixed with microcrystalline cellulose, cross-linked polyvinylpyrrolidone and magnesium stearate and compressed into tablet using appropriate tooling.

We claim:

1. A dry process for the preparation of valganciclovir hydrochloride solid dosage forms wherein the process comprises mixing amorphous valganciclovir hydrochloride with one or more pharmaceutically acceptable excipient(s) and forming into a solid dosage form.

2. The process according to claim 1 wherein the pharmaceutically acceptable excipient is one or more of filler, binder, disintegrant, glidant and lubricant.

3. The process according to claim 1 wherein the process comprises compacting valganciclovir hydrochloride alone or mixed with one or more of pharmaceutically acceptable excipient(s) by roller compactor or slugging; sizing the compacts or slugs into granules by milling; optionally mixing the granules with one or more of pharmaceutically acceptable excipients and forming a solid dosage form.

4. The process according to claim 3 wherein the compaction is done by roller compactor.

5. The process according to claims 3 wherein the solid dosage form is a tablet.

6. The process according to claim 3 wherein the solid dosage form is a capsule.

7. The process according to claim 1 wherein the mixture is directly compressed into a tablet.

8. The process according to claim 2 wherein the filler is one or more of microcrystalline cellulose, mannitol, sucrose, lactose, dextrose, calcium carbonate and sorbitol.

9. The process according to claim 2 wherein the binder is one or more of polyvinylpyrrolidone, hydroxypropyl cellulose, hydroxypropyl methylcellulose, starch and starch based binders, gelatin and gums.

10. The process according to claim 2 wherein the disintegrant is one or more of crospovidone, croscarmellose sodium, starch, hydroxypropylcellulose, hydroxypropylmethylcellulose, gums and sodium starch glycolate.

11. The process according to claim 2 wherein the glidant is one or more of talc and colloidal silicon dioxide.

12. The process according to claim 2 wherein the lubricant is one or more of magnesium stearate, stearic acid and sodium stearyl fumarate.

13. A solid dosage form comprising amorphous valganciclovir hydrochloride, filler, disintegrant, binder and lubricant prepared by the process of claim 1.

14. A solid dosage form comprising amorphous valganciclovir hydrochloride, microcrystalline cellulose, cross-linked polyvinylpyrrolidone, polyvinylpyrrolidone and magnesium stearate prepared by the process of claim 2.

15. The solid dosage form according to claim 13 wherein the solid dosage form is a tablet.

16. The solid dosage form according to claim 13 wherein the solid dosage form is a capsule.

17. A solid dosage form according to claim 13 wherein it additionally comprises another drug in a therapeutically effective amount.

18. A method of administering amorphous valganciclovir hydrochloride to a patient in need thereof as a solid dosage form prepared by a dry process wherein the process comprises mixing amorphous valganciclovir hydrochloride with one or more of pharmaceutically acceptable excipient(s) and forming into a solid dosage form.
